(19) United States
(12) Patent Application Publication
(10) Pub. No.: US 2026/0227343 A1
(43) Pub. Date: Aug. 6, 2026

(54) AUTOMATED VISUAL INSPECTION OF PREFILLED SYRINGES

(71) Applicant: AMGEN INC., Thousand Oaks, CA (US)

(72) Inventors: Al Patrick Goodwin, Dunleer (IE); Thomas Clark Pearson, Newbury Park, CA (US); Osvaldo Perez-Varela, Guaynabo, PR (US)

(21) Appl. No.: 19/147,818

(22) PCT Filed: Feb. 27, 2024
(86) PCT No.: PCT/US2024/017395

§ 371 Date: Jul. 14, 2025

Related U.S. Application Data
(60) Provisional application No. 63/487,313, filed on Feb. 28, 2023.

(51) Int. Cl. G01N 21/90; G01N 21/88; G06T 7/00

(52) U.S. Cl. CPC G01N 21/90; G01N 21/8803; G06T 7/0008; G06T 2207/10004; G06T 2207/30108

(57) ABSTRACT

A system for measuring an air gap in a syringe having a plunger and containing a fluid of the present disclosure may include one or more processors configured to control a syringe imaging device to capture a first image of at least a portion of the syringe with the syringe imaging device oriented at a first rotational angle around a central syringe axis of the syringe with respect to a first central imaging axis of the syringe imaging device. The one or more processors may also be configured to control the syringe imaging device to capture a second image of at least a portion of the syringe with the syringe imaging device oriented at a second rotational angle around the central syringe axis with respect to a second central imaging axis. The one or more processors may be further configured to determine an air gap measurement between the plunger and fluid by analyzing at least the first image and the second image.

### CROSS-REFERENCE TO RELATED APPLICATIONS

Priority is claimed to U.S. Provisional Patent Application No. 63/447,313, filed Feb. 28, 2023, the entire contents of which are hereby incorporated by reference herein.

### FIELD OF DISCLOSURE

The present application relates generally to the inspection of vessels, and more specifically to imaging systems for automated visual inspection of prefilled syringes.

### BACKGROUND

In certain contexts, such as quality control procedures for manufactured drug products, it is necessary to examine vessels (e.g., prefilled syringes, containers, vials, cartridges, etc., and/or their contents) for the presence of various defects (e.g., air gap measurements, plunger depth measurements, cracks, defective seals, low fill, high fill, foreign particles, fibers, etc.). The acceptability of a given vessel or sample, under the applicable quality standards, may depend on metrics such as a condition of the vessel, the presence of undesired particles within the vessel, etc.

Plunger depth and air gap are two important quality parameters that can affect combination product functionality, shelf life, and sterility of the product in a syringe filled with fluid (e.g., a drug product, etc.). If the prefilled syringe has unacceptable metrics, the syringe and the contents may be rejected and discarded.

Known measurement techniques can be grouped into three general categories: manual techniques, electronic sensor-based techniques, and machine vision-based techniques. Manual techniques include handheld or manually operated equipment like calipers, depth gauges, and optical comparators. Plunger depth and air gap have traditionally been measured with calipers. Calipers are inexpensive and easy to use, which allows for high speed and measurement versatility, but measurement using calipers places a heavier burden on human operators to ensure accuracy and consistency. Depth gauges have been considered for select plunger depth measurements, but have similar drawbacks as calipers, as well as an added risk of moving the plunger during measurement. In contrast, an optical comparator is both accurate and repeatable across users and is widely considered a preferred standard for plunger depth measurements. Drawbacks of the optical comparator include its slow speed, impending obsolescence, and potentially destructive nature due to light intensity required to produce a silhouette image of the syringe.

Electronic sensor techniques include electronic components capable of measuring distance (e.g., a confocal microscope, an optical comparator, ultrasonic sensors, etc.). While a distance sensor may be acceptable to measure plunger depth, a distance sensor is not able to measure an air gap. Distance sensors utilize an optical beam of infrared or visible light that is reflected off a surface. The distance sensor detects the reflected light and, depending on the underlying measurement principle, converts a time, wavelength, or angle of reflection into a distance measurement. Plunger depth may then be calculated by subtracting a distance between a syringe flange and the sensor, and a plunger and the sensor.

Machine vision techniques include acquiring an image of the syringe and digitally determining air gap or plunger depth via a computer algorithm. To handle the quantities of prefilled syringes typically associated with commercial production of pharmaceuticals, product inspection tasks (e.g., plunger depth measurements, air gap measurements, etc.) have increasingly become automated. Known automated visual inspection (AVI) systems (e.g., system 100 of FIG. 1) have struggled to overcome various barriers to achieving good product fidelity void of system complexities. The system 100 includes a camera 126 with a central imaging axis 128 aligned with a central syringe axis 103 of a prefilled syringe 105 and an illumination source 131. The prefilled syringe 105 may contain a plunger 110 and a fluid 120. The camera 126 captures a single image 101 of a portion of the prefilled syringe 105 with the central imaging axis 128 aligned with the central syringe axis 103 from a fixed, predetermined rotational angle around the central syringe axis 103, and may measure a plunger depth 185 and/or an air gap 180 between the plunger 110 and the fluid 120 based on the single image 101. Measurement of an air gap 180 presents issues for system 100 in fluid 120 often collects at the junction of the plunger 110 and an interior of a sidewall 114 of the syringe 105, making the measurement point 114 difficult to locate. Liquid droplets and/or air bubbles at the junction of the plunger 110 and syringe sidewall 114 often occlude the measurement point for air gap 180. Thus, one of the main difficulties with measuring air gap 180 is properly locating the junction of the stopper and syringe sidewall 114. Furthermore, a meniscus of a top surface of a drug solution is sometime asymmetrical and difficult to locate accurately when based on a single image 101.

Accordingly, AVI systems are needed that more accurately measure an air gap in a pre-filled syringe.

### SUMMARY

Embodiments described herein relate to fixed-position imaging systems and automated visual inspection (AVI) systems that incorporate fixed-position imaging.

As described herein, a computer-implemented method for measuring an air gap in a syringe having a plunger and containing a fluid includes capturing, using a syringe imaging device, a first image of at least a portion of the syringe from a first rotational angle around a central syringe axis of the syringe with respect to a first central imaging axis of the syringe imaging device. The method also includes capturing, using the syringe imaging device, a second image of at least a portion of the syringe from a second rotational angle around the central syringe axis with respect to a second central imaging axis of the syringe imaging device. The method further includes determining, using one or more processors, an air gap measurement between the plunger and fluid by analyzing at least the first image and the second image.

A system for measuring an air gap in a syringe having a plunger and containing a fluid includes a syringe imaging device and one or more processors. The one or more processors is configured to control a syringe imaging device to capture a first image of at least a portion of the syringe from a first rotational angle around a central syringe axis of the syringe with respect to a first central imaging axis of the syringe imaging device. The one or more processors is also configured to control the syringe imaging device to capture a second image of at least a portion of the syringe from a second rotational angle around the central syringe axis with respect to a second central imaging axis of the syringe imaging device. The one or more processors is further configured to determine an air gap measurement between the plunger and fluid by analyzing at least the first image and the second image.

A non-transitory computer-readable medium storing computer-readable instructions that, when executed by one or more processors, causes the one or more processors to control a syringe imaging device to capture a first image of at least a portion of the syringe from a first rotational angle around a central syringe axis of the syringe with respect to a first central imaging axis of the syringe imaging device. Further execution of the computer-readable instructions by the one or more processors, also causes the one or more processors to control the syringe imaging device to capture a second image of at least a portion of the syringe from a second rotational angle around the central syringe axis with respect to a second central imaging axis of the syringe imaging device. Further execution of the computer-readable instructions by the one or more processors, further causes the one or more processors to determine an air gap measurement between the plunger and fluid by analyzing at least the first image and the second image.

Novel AVI systems are provided that more accurately measure an air gap in a pre-filled syringe.

### BRIEF DESCRIPTION OF THE DRAWINGS

The skilled artisan will understand that the figures described herein are included for purposes of illustration and do not limit the present disclosure. The drawings are not necessarily to scale, and emphasis is instead placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 depicts a side profile view of an imaging system of the prior art.

FIG. 2 depicts an example prefilled syringe with a plunger, a fluid, and an air gap.

FIG. 3A depicts an example automated visual inspection (AVI) system including an example desktop automated syringe inspection (DASI) system.

FIG. 3B depicts an interior view of the example desktop automated syringe inspection (DASI) system of FIG. 3A.

FIG. 3C depicts an expanded view of an example syringe rotation mechanism with glass tube syringe fixture of the example desktop automated syringe inspection (DASI) system of FIG. 3A.

FIG. 4A depicts an example calibration standard.

FIG. 4B depicts the example calibration standard of FIG. 4A inserted into the example desktop automated syringe inspection (DASI) system of FIGS. 3A-3C.

FIG. 5 depicts a high-level block diagram of the example desktop automated syringe inspection (DASI) system of FIGS. 3A-3C.

FIG. 6 depicts an example method of calibrating a desktop automated syringe inspection (DASI) system.

FIG. 7 depicts an example method of operating a desktop automated syringe inspection (DASI) system.

FIG. 8 depicts an example method of generating air gap measurement data.

FIG. 9 depicts an example method of generating aggregate air gap measurement data.

FIG. 10 depicts an example method of generating plunger depth measurement data.

FIG. 11 depicts an example method of generating aggregate plunger depth measurement data.

FIG. 12 depicts an example automated visual inspection (AVI) system configured to implement techniques of this disclosure.

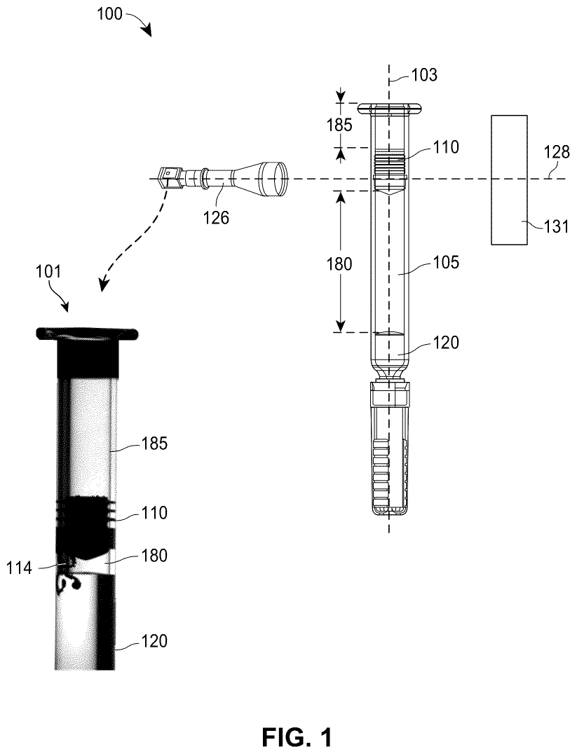

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercial feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

The systems and methods described herein measure an air gap (and possibly also a plunger depth and/or other characteristics) in a prefilled syringe that has a plunger and contains a fluid by capturing a first image of at least a portion of the syringe with the syringe imaging device oriented at a first rotational angle around a central syringe axis of the syringe, and capturing a second image of at least a portion of the syringe with the syringe imaging device oriented at a second rotational angle around the central syringe axis. The systems and methods measure at least an air gap by analyzing at least the first image and the second image.

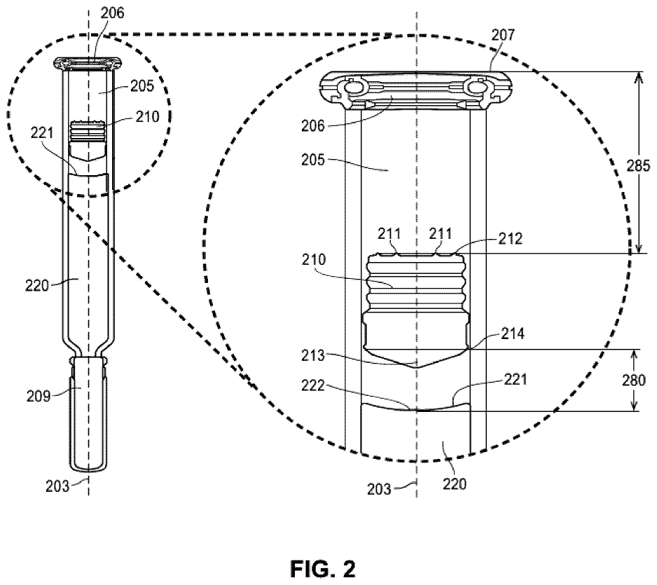

FIG. 2 illustrates a typical prefilled syringe 205 filled with a fluid 220 and with a plunger 210 inserted. A plunger depth 285 is defined as a difference between a topmost point 207 of a syringe flange 206 and a topmost point 212 on the plunger 210 not including the small lugs, or dimples 211. A top surface of the flange 206 is rarely planer and, therefore, any portion of the flange 206 that is determined to be furthest away from the plunger 210 may be determined to be the topmost point 207. An air gap 280 is a measurement between a bottom 222 of a meniscus curve 221 that defines a top surface of the fluid 220 and a point 214 where a bottom 213 of the plunger 210 meets an inner surface of the syringe 205. Measurement of air gap 280 is performed with a central syringe axis 203 of the syringe 205 oriented vertically and a syringe needle 209 oriented downward with respect to the syringe flange 206 so that air rises upward through the fluid 220 and forms the air gap 280.

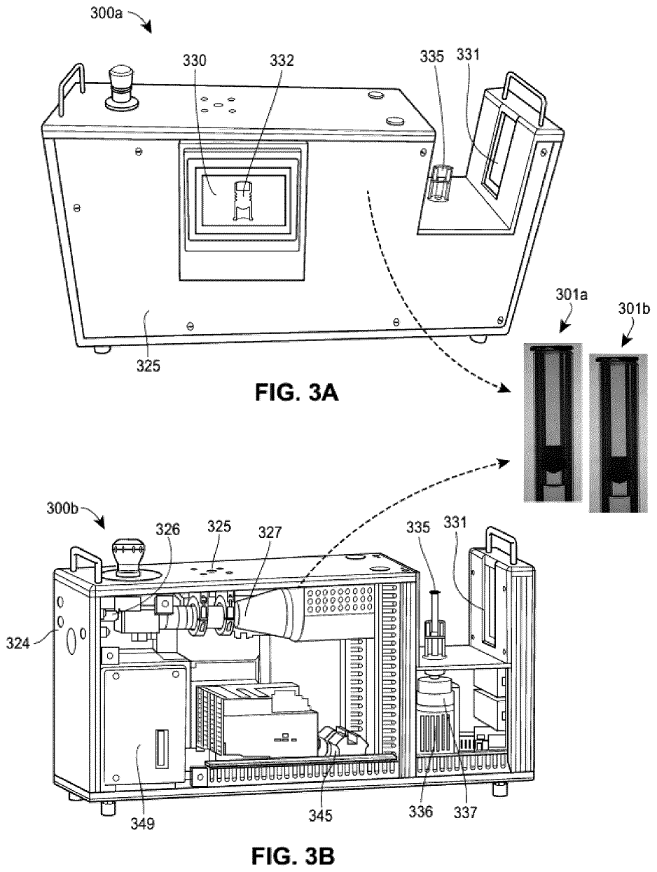

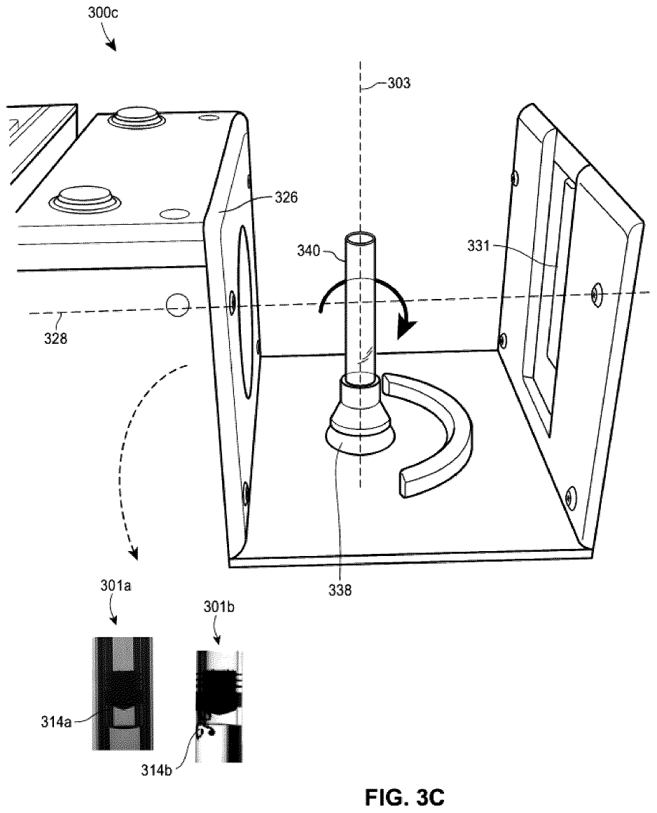

FIGS. 3A-3C depict an automated visual inspection (AVI) system 300a,b that includes a desktop automated syringe inspection (DASI) system 325 configured to provide an air gap measurement (e.g., measurement 280), and possibly a plunger depth measurement (e.g., measurement 285), for a prefilled syringe 205. The DASI system 325 includes at least one external port 324 configured to output an aggregate air gap measurement based on an analysis of at least two different images 301a,b. The at least one external port 324 may be further configured to output an aggregate plunger depth measurement based on an analysis of at least two different images 301a,b.

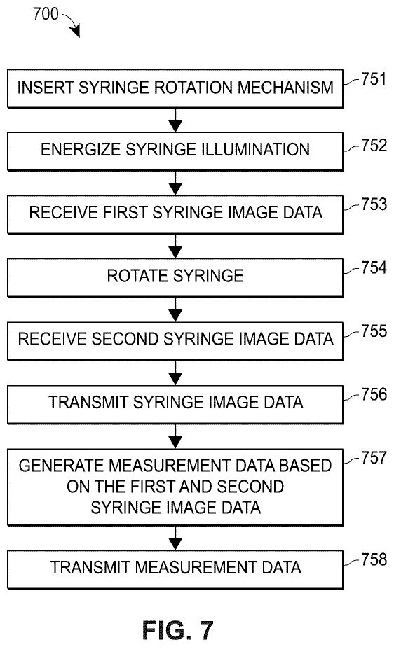

The external port 324 outputs the aggregate air gap measurement 180 in a standard format (e.g., an electronic batch record (EBR) system format, a qualified GMP part 11 data management and storage system format, etc.). The external port 324 may also output the aggregate plunger depth measurement 185 in the standard format. The DASI system 325 may further include a user interface 330. The user interface 330 may include a touch screen human machine interface (HMI) 332. A method 900 of generating an aggregate air gap measurement is described with reference to FIG. 9. A method 1100 of generating an aggregate plunger depth measurement is described with reference to FIG. 11. A method 700 of calibrating a DASI system is described with reference to FIG. 7.

The DASI system 325 also includes a camera 326 (e.g., a five-megapixel camera, etc.) with a telecentric lens 327, a syringe fixture 335, a programmable logic controller (PLC) 345, and an image processing module 349. The syringe fixture 335 is designed to prevent a syringe needle 209 from bending when a prefilled syringe 205 is inserted into the syringe fixture 335. The syringe fixture 335 may enable an operator to insert a prefilled syringe 205 into the syringe fixture 335 with one hand.

The syringe fixture 335 may include a glass tube that has an inner diameter slightly larger than an outside diameter of a given syringe (such as the syringe 205). The syringe may be inserted into the glass tube such that the syringe is held in a stable vertical position and does not occlude any part of the syringe. Accordingly, an air gap and plunger depth can be measured at any fill level. Additionally, or alternatively, an air gap and plunger depth can be measured in a syringe with a fill level of less than 0.33 ml.

The DASI system 325 further include a motor 336 and a torque limiting clutch 337. The motor 336 and a torque limiting clutch 337 may be configured to rotate the syringe axially. The camera 326 may be configured to capture a series of images 301a,b while the syringe makes a complete rotation about a central syringe axis 303. The camera 326 may be configured to capture a series of at least twenty-five images per revolution. The DASI system 325 may generate an aggregate air gap measurement based on an analysis of at least twenty-five images. Analysis of at least twenty-five images increases a likelihood that a syringe-sidewall junction 314a,b is accurate compared to analysis of fewer images and, moreover, when compared to analysis of a single image 101. Notably, the syringe-sidewall junction 314b is at least partially obscured in the image 301b. Accordingly, an air gap measurement based only on image 301b would have a higher likelihood of being erroneous compared to an air gap measurement based on image 301a.

As the syringe rotates axially, the plunger depth and air gap may be measured by, for example, using any suitable image processing technique(s) (e.g., edge detection within an image, pixel location within an image, etc.). The plunger depth and air gap measurements may be transmitted to the PLC 345, where each measurement for a particular syringe may be stored. Upon taking all of the images 301a,b during a complete rotation, the PLC 345 may perform non-linear filtering by removing a small number of extreme measurements. For example, a largest and a smallest three air gap measurements may be removed from a set of measurement data (e.g., a set of measurement data generated from analysis of at least twenty-five images 301a,b). Thereby, when a droplet obscures the syringe-sidewall junction 314b, the associated air gap measurement could either be excessively large or small compared to an actual value. While removing the three largest and three smallest air gap measurements may be performed, however, other quantities of measurements might give better or worse results depending on the fluid 220. After non-linear filtering of the measurement data, the PLC 345 may average the remaining measurements to generate an aggregate air gap measurement.

An aggregate plunger depth measurement may be similarly generated using multiple measurements while the syringe is rotating, which also can improve accuracy. The plunger depth measurement begins at the highest point on the syringe flange. This point can be difficult to find from just one side-perspective of the syringe. By detecting the highest point on several images as the syringe rotates enables slightly more accurate plunger depth measurements. Likewise, the top edge of the plunger, or a plane that is defined by a base of the dimples 211, or lugs, on top of the plunger 210 is frequently difficult to accurately detect due to different presentations of the dimples 211 on the plunger. By taking an average of several plunger top positions, the DASI system 325 increases accuracy of plunger depth measurement compared to analysis of only one image.

As a quality control check of the DASI system 325, a set of images (e.g., at least twenty-five images) may be compared to ensure that the syringe actually rotated. If an error occurs with motor communications or power transfer hardware, the set of images 301a,b may have a similar appearance to one another since there would be no syringe movement between the images. Thereby, the DASI system 325 may determine that, for example, the torque limiting clutch 337 between the motor 336 and syringe fixture 335 may have disengaged. On the other hand, if the motor rotated the syringe properly, there should be differences in each image, particularly around the flange area.

In some embodiments, this type of check includes using a separate processor to monitor the motor encoder signals and thereby confirm movement. However, this brings some additional cost and complexity to the system. It also does not ensure that the clutch and shaft couplings between the motor and syringe are working properly. Thus, in other embodiments, the already-acquired images are used to provide a more robust check of proper syringe rotation.

The DASI system 325 may also include an illumination source 331. The illumination source 331 may be configured as a near infrared (NIR) emitting backlight. Because this is generally a non-destructive test, the light exposure is preferably kept at a minimum so that drug product quality is not compromised. Light exposure is mitigated by strobing the light only while images are being acquired. This causes a sequence of 20 to 30 or more short flashes of light. This flashing, or blinking, of the lights in rapid succession can be annoying to an operator and can be a trigger for some seizures. As such, an NIR light may be chosen, as this is not visible to humans and causes less product damage than visible light does.

Preferably, the hardware configuration of the DASI system 325 is compact in order to facilitate movement, and self-contained so that all of the image acquisition and processing are performed therein. The DASI system 325 may use high-quality optics in the form of a telecentric lens to minimize spatial distortions and parallax encountered with standard lenses. Moreover, the DASI system 325 may be configured to quickly and easily load a syringe into fixturing, such that measurements can be made in a manner that is repeatable, easy, ergonomic, and enables quick insertion and removal of a syringe (so as to minimize time needed to measure several syringes).

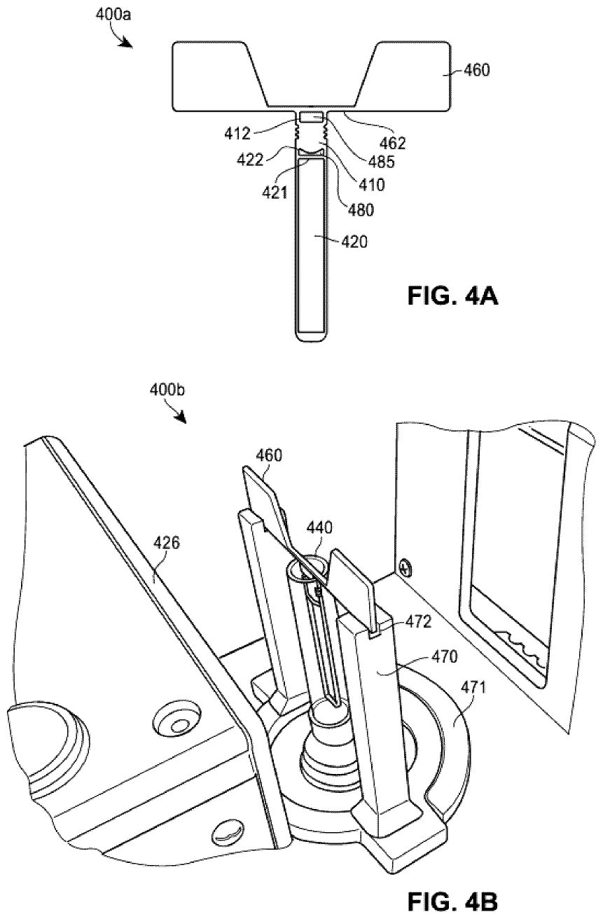

FIGS. 4A and 4B show a DASI calibration device 400a,b including a calibration standard 460 and a fixture 470. The calibration standard 460 includes a topmost point of a syringe flange 462, and a topmost point 412 on a plunger 410 that define a predetermined plunger depth measurement 485. The calibration standard 460 also includes a bottom 422 of a meniscus curve that defines a top surface of a fluid 420, and a point 421 where a bottom of the plunger 410 meets an inner surface of the syringe that define a predetermined air gap measurement 480. The calibration standard 460 may be fabricated by laser cutting thin metal to form a two-dimensional representation of a prefilled syringe 205.

The fixture 470 is secured in a fixed position via a base 471. The base 471 is configured to prevent the fixture from rotating. The fixture 470 also includes calibration standard receptacle 472. The fixture 470 is configured to prevent the calibration standard 460 from rotating when a portion of the calibration standard 460 is received within the calibration standard receptacle 472. The fixture 470 may include a syringe receptacle 440 (e.g., a glass tube).

A DASI 300a-c may be calibrated based on an image of the calibration standard 460 inserted within the syringe receptacle 440 and held in a fixed position via the fixture 470. Further details of a method to calibrate a DASI are described with respect to FIG. 6.

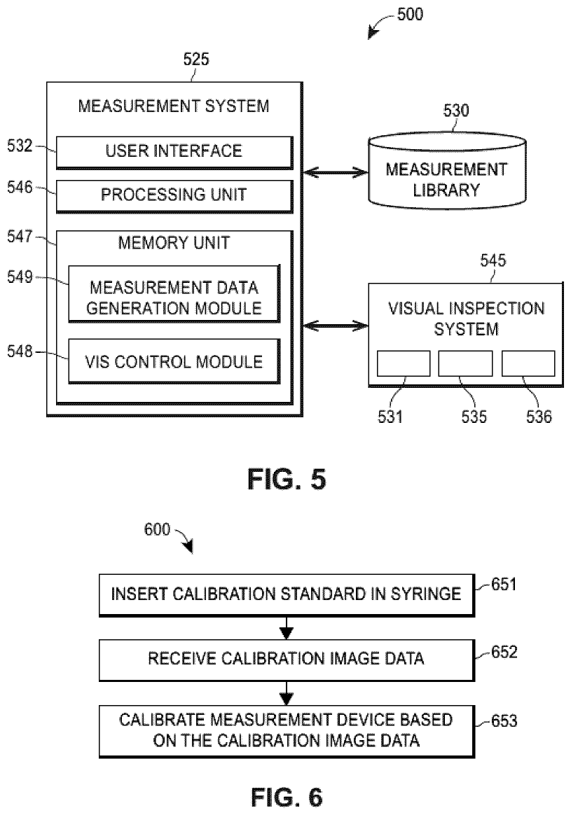

FIG. 5 is a high-level block diagram of a desktop automated syringe inspection (DASI) system 500 that may implement various techniques relating to the training (and possibly validation and/or qualification) and/or use of one or more neural networks or other non-machine learning (ML) system to measure a plunger depth and/or an air gap. The DASI system 500 could also be used to test/qualify non-ML AVI systems. In addition to, or as an alternative to, ML systems, the DASI system 500 may include “computer vision” algorithms that do not use ML, but instead use fixed rules (e.g., empty vial, low fill, high fill, etc.). The measurement system 525 outputs aggregate air gap measurement data, via an external port 324, into an Electronic Batch Record (EBR) system without any software modifications to the EBR system. The measurement system 525 may also output aggregate plunger depth, via the external port 324, into the Electronic Batch Record (EBR) system without any software modifications to the EBR system.

A DASI system 500 may include, for example, one or more automated visual inspection (AVI) neural network(s). Once trained and qualified, the DASI system 500 may be used in production to detect defects associated with vessels and/or contents of those vessels. In a pharmaceutical context, for example, the DASI system 500 may be used to detect defects associated with syringes, cartridges, vials or other vessel types (e.g., bruised crimps/seals, cracks, scratches, stains, missing components, etc., of the vessels), and/or to detect defects associated with liquid or lyophilized drug products within the vessels (e.g., the presence of fibers, metallic particles, and/or other foreign particles, variations in color of the product, etc.). As used herein, “defect detection” may refer to the classification of vessel images as exhibiting or not exhibiting defects (or particular defect categories), and/or may refer to the detection of particular objects or features (e.g., particles or cracks) that are relevant to whether a vessel and/or its contents should be considered defective, depending on the embodiment.

DASI system 500 includes a visual inspection system (VIS) 545 communicatively coupled to a measurement system 525. VIS 545 includes hardware (e.g., a light source 531, syringe fixture 535, syringe rotation mechanism motor 536, etc.), as well as firmware and/or software, that is configured to capture digital images of a sample (e.g., a prefilled syringe holding a fluid or lyophilized substance). VIS 545 may include any of the imaging systems described herein respectively with reference to FIGS. 2-4, for example, or may be some other suitable VIS.

For ease of explanation, DASI system 500 is described herein as training and validating one or more AVI neural networks using vessel images 301a and 301b from VIS 545, and then using the trained/validated neural network(s) to perform AVI/defect detection. It is understood, however, that this need not be the case. For example, the DASI system 500 may perform training and/or validation using vessel images generated by a number of different visual inspection systems instead of, or in addition to, VIS 545. Moreover, the training/validation may be performed by another system, and DASI system 500 may then use the trained neural network(s) (e.g., during commercial production). In some embodiments, some or all of the vessel images used for training and/or validation are generated using one or more offline (e.g., lab-based) “mimic stations” that closely replicate important aspects of commercial line equipment stations (e.g., optics, lighting, etc.), thereby expanding the training and/or validation library without causing excessive downtime of the commercial line equipment.

VIS 545 may image each of a number of vessels simultaneously. To this end, VIS 545 may include, or operate in conjunction with, holding means such as a conveyance mechanism, a turntable, a cartesian robot, carousel, starwheel and/or any other holding means that can successively move each vessel into an appropriate position for imaging, and then moves the vessel away once imaging of the vessel is complete. While not shown in FIG. 5, VIS 545 may include a communication interface and processors to enable communication with measurement system 525. In other embodiments (e.g., lab-based setups), the VIS 545 includes simpler holding means (e.g., a stage with a hole covered by a glass plate).

Measurement system 525 may generally be configured to control/automate the operation of VIS 545, and to receive and process images captured/generated by VIS 545, as discussed further below. Measurement system 525 may be a general-purpose computer that is specifically programmed to perform the operations discussed herein, or may be a special-purpose computing device. As seen in FIG. 5, measurement system 525 includes a user interface 532, a processing unit 546, and a memory unit 547. In some embodiments, however, measurement system 525 includes two or more computers that are either co-located or remote from each other. In these distributed embodiments, the operations described herein relating to processing unit 546 and memory unit 547 may be divided among multiple processing units and/or memory units, respectively.

Processing unit 546 includes one or more processors, each of which may be a programmable microprocessor that executes software instructions stored in memory unit 547 to execute some or all of the functions of measurement system 525 as described herein. Processing unit 546 may include one or more graphics processing units (GPUs) and/or one or more central processing units (CPUs), for example. Alternatively, or in addition, some of the processors in processing unit 546 may be other types of processors (e.g., application-specific integrated circuits (ASICs), field-programmable gate arrays (FPGAs), etc.), and some of the functionality of computer system 604 as described herein may instead be implemented in hardware.

Memory unit 547 may include one or more volatile and/or non-volatile memories. Any suitable memory type or types may be included in memory unit 547, such as read-only memory (ROM), random access memory (RAM), flash memory, a solid-state drive (SSD), a hard disk drive (HDD), and so on. Collectively, memory unit 547 may store one or more software applications, the data received/used by those applications, and the data output/generated by those applications.

Memory unit 547 stores the software instructions of various modules that, when executed by processing unit 546, performs various functions for the purpose of training, validating, and/or qualifying one or more AVI neural networks. Specifically, in the example embodiment of FIG. 5, memory unit 547 includes a measurement data generation module 549 and a visual inspection system (VIS) control module 526. In other embodiments, memory unit 547 may omit one or more of modules 548, 549 and/or include one or more additional modules. In addition, or alternatively, one, some, or all of modules 548, 549 may be implemented by a different computer system (e.g., a remote server coupled to measurement system 525 via one or more wired and/or wireless communication networks). Moreover, the functionality of any one of modules 548 and 549 may be divided among different software applications and/or computer systems. As just one example, in an embodiment where measurement system 525 accesses a web service to train and use one or more AVI neural networks, the software instructions of measurement data generation module 549 may be stored at a remote server.

Measurement data generation module 549 comprises software that uses images stored in an image library 530 to train one or more AVI neural networks. Image library 530 may be stored in memory unit 547, or in another local or remote memory (e.g., a memory coupled to a remote library server, etc.). In addition to training, module 549 may implement/run the trained AVI neural network(s), e.g., by applying images newly acquired by VIS 545 (or another visual inspection system) to the neural network(s), possibly after certain pre-processing is performed on the images as discussed below. In various embodiments, the AVI neural network(s) trained and/or run by module 549 may classify entire images (e.g., defect vs. no defect, or presence or absence of a particular type of defect such as a crimp bruise or crimp defect generally, etc.), detect objects in images (e.g., detect the position of foreign objects that are not bubbles within vessel images), or some combination thereof (e.g., one neural network classifying images, and another performing object detection). As used herein, unless the context clearly indicates a more specific use, “object detection” broadly refers to techniques that identify the particular location of an object (e.g., a particle, a fiber, etc.) within an image, and/or that identify the particular location of a feature of a larger object (e.g., a bruised crimp or seal, a crack or chip on a syringe or cartridge barrel, etc.), and can include, for example, techniques that perform segmentation of the vessel image or image portion (e.g., pixel-by-pixel classification), or techniques that identify objects and place bounding boxes (or other boundary shapes) around those objects.

In embodiments where the AVI neural network(s) detect vessel defects, the defects may relate to any suitable vessel feature(s). Referring to the example vessels of FIGS. 2-4B, for instance, a particular AVI neural network implemented by the measurement data generation module 549 may detect whether a vessel has a crack or stain, whether flange is misshapen, whether needle shield is not properly positioned, whether plunger or piston has any defects, whether luer lock 528 has any defects, whether a crimp is properly positioned and/or has any defects (e.g., bruising), whether a flip cap is properly positioned and/or has any defects, and so on.

Module 549 may run the trained AVI neural network(s) for purposes of validation, qualification, and/or inspection during commercial production. In one embodiment, for example, module 549 is used only to train and validate the AVI neural network(s), and the trained neural network(s) is/are then transported to another computer system for qualification and inspection during commercial production (e.g., using another module similar to module 549). In some embodiments where measurement data generation module 549 trains/runs multiple neural networks, the module 549 includes separate software for each neural network.

AVI neural network training may be performed on images from, for example, six vials after augmenting the associated training images by adjusting brightness, vertical mirroring, adding noise, and skewing the images, as well as skewing the bounding boxes (i.e., the training set may be multiplied fivefold). Generally, deep learning may be used to detect defects in the images. Use of previously trained AVI neural network(s) further reduces time required to set up an automated inspection recipe for new products. AVI neural networks of the present disclosure may be implemented for high-mix, low-volume production scenario such as clinical operations or small batches of product. then using modern deep learning techniques (e.g., measurement data generation module 549 of FIG. 5).

In some embodiments, VIS control module 548 controls/automates operation of VIS 545 such that vessel images can be generated with little or no human interaction. VIS control module 525 may cause a given fixed-position imaging system to capture a vessel image by sending a command or other electronic signal (e.g., generating a pulse on a control line, etc.) to that imager. VIS 545 may send the captured vessel images to measurement system 525, which may store the images in memory unit 547 for local processing. In alternative embodiments, VIS 545 may be locally controlled, in which case VIS control module 525 may have less functionality than is described herein (e.g., only handling the retrieval of images from VIS 545), or may be omitted entirely from memory unit 547.

FIG. 6 is a method 600 of calibrating a desktop automated syringe inspection (DASI) system, which may be implemented by a processor (e.g., processing unit 546 of FIG. 5) executing, for example, at least a portion of VIS control module 548 and/or the measurement data generation module 549. The DASI system may be similar to, for example, any one of the DASI systems 325 of FIG. 3A-3B or 500 of FIG. 5. In particular, a calibration standard 400a is inserted into a syringe receptacle 440 of a syringe rotation mechanism 338 with a central syringe axis 303 aligned with a central imaging axis 328 (block 651).

The processing unit 546 may execute the VIS control module 548 to cause the processing unit 546 to, for example, receive calibration image data from an imaging device 426 (block 652). The processing unit 546 may execute the measurement data generation module 549 to cause the processing unit 546 to, for example, calibrate the DASI system 325 (block 653). For example, the processing unit 546 may receive the predetermined air gap measurements 480 and plunger depth measurements 485. The processing unit 546 may calibrate the DASI system 325 by comparing an air gap measurement determined based on an analysis of calibration image data with the predetermined air gap measurement 480. Additionally, or alternatively, the processing unit 546 may calibrate the DASI system 325 by comparing a plunger depth measurement determined based on an analysis of calibration image data with the predetermined plunger depth measurement 485.

FIG. 7 is a method 700 of operating a DASI system 325, which may be implemented by the processing unit 546 executing, for example, at least a portion of VIS control module 548 and/or the measurement data generation module 549. The DASI system may be similar to, for example, any one of the DASI systems 325 of FIG. 3A-3B or 500 of FIG. 5. In particular, a prefilled syringe 205 is inserted into a syringe receptacle 340 (e.g., a glass tube) of a syringe rotation mechanism 338 with a central syringe axis 303 aligned with a central imaging axis 328 (block 751).

The processing unit 546 may execute the VIS control module 548 to cause the processing unit 546 to, for example, energize an illumination source 310 (block 752). The processing unit 546 may further execute the VIS control module 548 to cause the processing unit 546 to, for example, receive first syringe image data 301a from the imaging device 326 (block 753).

The processing unit 546 may further execute the VIS control module 548 to cause the processing unit 546 to, for example, control the syringe rotation mechanism 338 to rotate the syringe 205 around the central syringe axis 303 (block 754). The processing unit 546 may further execute the VIS control module 548 to cause the processing unit 546 to, for example, receive second syringe image data 301b from the imaging device 326 (block 755). The processing unit 546 may further execute the VIS control module 548 to cause the processing unit 546 to, for example, transmit the first syringe image data 301a and the second syringe image data 301b to the measurement library 530 (block 756).

The processing unit 546 may execute the measurement data generation module 549 to cause the processing unit 546 to, for example, generate measurement data (e.g., a topmost point 207 of a syringe flange 206, a topmost point 212 on the plunger 210 not including the small lugs, a bottom 222 of a meniscus curve 221 that defines a top surface of the fluid 220, a point 214 where a bottom 213 of the plunger 210 meets an inner surface of the syringe 205, air gap measurement data, plunger depth measurement data, etc.) based on the first syringe image data 301a and the second syringe image data 301b (block 757). A plunger depth measurement 285 may be a difference between a topmost point 207 of a syringe flange 206 and a topmost point 212 on the plunger 210 not including the small lugs, or dimples 211. An air gap measurement 280 may be a difference between a bottom 222 of a meniscus curve 221 that defines a top surface of the fluid 220 and a point 214 where a bottom 213 of the plunger 210 meets an inner surface of the syringe 205.

The processing unit 546 may further execute the VIS control module 548 to cause the processing unit 546 to, for example, transmit the measurement data from the VIS system to the measurement system 525 and/or the measurement library 530 (block 758).

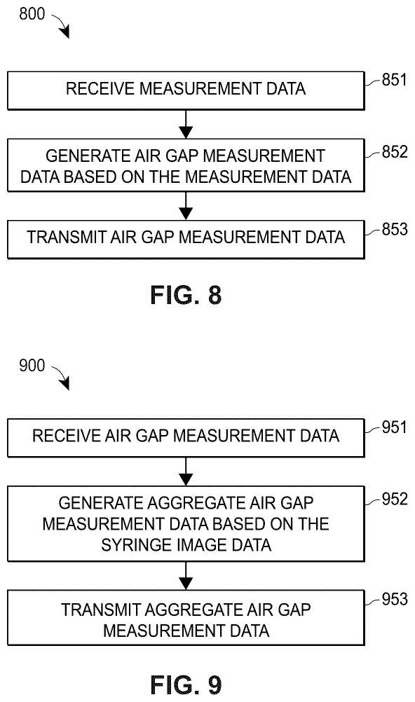

FIG. 8 is a method 800 of operating a DASI system, which may be implemented by the processing unit 546 executing, for example, at least a portion of VIS control module 548 and/or the measurement data generation module 549. The DASI system may be similar to, for example, any one of the DASI systems 325 of FIG. 3A-3B or 500 of FIG. 5. In particular, the processing unit 546 may execute the VIS control module 548 to cause the processing unit 546 to, for example, receive measurement data (block 851). The measurement data may include a bottom 222 of a meniscus curve 221 that defines a top surface of the fluid 220 and a point 214 where a bottom 213 of the plunger 210 meets an inner surface of the syringe 205.

The processing unit 546 may execute the measurement data generation module 549 to cause the processing unit 546 to, for example, generate air gap measurement data based on the measurement data (block 852). A first air gap measurement 280 may be a difference between a bottom 222 of a meniscus curve 221 that defines a top surface of the fluid 220 and a point 214 where a bottom 213 of the plunger 210 meets an inner surface of the syringe 205 based on an analysis of a first image 301a. A second air gap measurement 280 may be a difference between a bottom 222 of a meniscus curve 221 that defines a top surface of the fluid 220 and a point 214 where a bottom 213 of the plunger 210 meets an inner surface of the syringe 205 based on an analysis of a second image 301b.

The processing unit 546 may further execute the VIS control module 548 to cause the processing unit 546 to, for example, transmit the air gap measurement data from the VIS 545 to the measurement system 525 or the measurement library 530 (block 853).

FIG. 9 is a method 900 of operating a DASI system, which may be implemented by the processing unit 546 executing, for example, at least a portion of the visual inspection system (VIS) control module 548 and/or the measurement data generation module 549. The DASI system may be similar to, for example, any one of the DASI systems 325 of FIG. 3A-3B or 500 of FIG. 5. In particular, the processing unit 546 may execute the VIS control module 548 to cause the processing unit 546 to, for example, receive air gap measurement data (block 951). The air gap measurement data may include a first air gap measurement based on an analysis of a first image 301a and a second air gap measurement based on an analysis of a second image 301b.

The processing unit 546 may execute the measurement data generation module 549 to cause the processing unit 546 to, for example, generate aggregate air gap measurement data based on the air gap measurement data (block 952). The aggregate air gap measurement data may be based on the first air gap measurement and the second air gap measurement. For example, the aggregate air gap measurement data may be an average of the first air gap measurement and the second air gap measurement.

The processing unit 546 may further execute the VIS control module 548 to cause the processing unit 546 to, for example, transmit the aggregate air gap measurement data to the measurement library 530 (block 953).

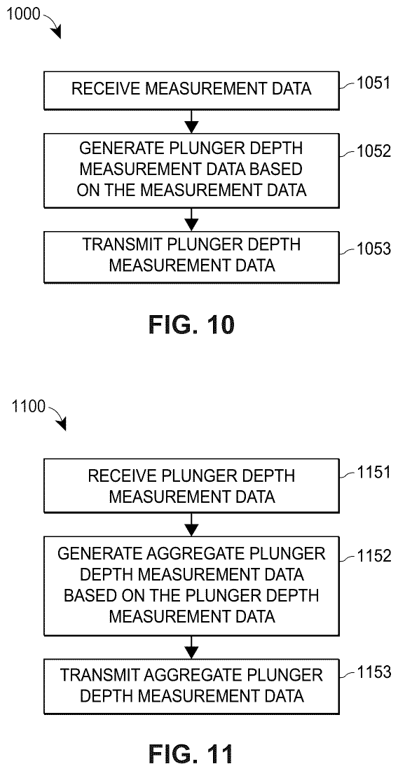

FIG. 10 is a method 1000 of operating a DASI system, which may be implemented by the processing unit 546 executing, for example, at least a portion of the visual inspection system (VIS) control module 548 and/or the measurement data generation module 549. The DASI system may be similar to, for example, any one of the DASI systems 325 of FIG. 3A-3B or 500 of FIG. 5. In particular, the processing unit 546 may execute the VIS control module 548 to cause the processing unit 546 to, for example, receive measurement data (block 1051). The measurement data may include a topmost point 207 of a syringe flange 206 and a topmost point 212 on the plunger 210 not including the small lugs.

The processing unit 546 may execute the measurement data generation module 549 to cause the processing unit 546 to, for example, generate plunger depth measurement data based on the measurement data (block 1052). A first plunger depth measurement 285 may be a difference between a topmost point 207 of a syringe flange 206 and a topmost point 212 on the plunger 210 not including the small lugs, or dimples 211 based on an analysis of a first image 301a. A second plunger depth measurement 285 may be a difference between a topmost point 207 of a syringe flange 206 and a topmost point 212 on the plunger 210 not including the small lugs, or dimples 211 based on an analysis of a second image 301b.

The processing unit 546 may further execute the VIS control module 548 to cause the processing unit 546 to, for example, transmit the plunger depth measurement data from the VIS 545 to the measurement system 525 or the measurement library 530 (block 1053).

FIG. 11 is a method 1100 of operating a DASI system, which may be implemented by the processing unit 546 executing, for example, at least a portion of VIS control module 548 and/or the measurement data generation module 549. The DASI system may be similar to, for example, any one of the DASI systems 325 of FIG. 3A-3B or 500 of FIG. 5. In particular, the processing unit 546 may execute the VIS control module 548 to cause the processing unit 546 to, for example, receive plunger depth measurement data (block 1151). The plunger depth measurement data may include a first plunger depth measurement based on an analysis of a first image 301a and a second plunger depth measurement based on an analysis of a second image 301b.

The processing unit 546 may execute the measurement data generation module 549 to cause the processing unit 546 to, for example, generate aggregate plunger depth measurement data based on the plunger depth measurement data (block 1152). The aggregate plunger depth measurement data may be based on the first plunger depth measurement and the second plunger depth measurement. For example, the aggregate plunger depth measurement data may be an average of the first plunger depth measurement and the second plunger depth measurement.

The processing unit 546 may further execute the VIS control module 548 to cause the processing unit 546 to, for example, transmit the aggregate plunger depth measurement data from the VIS 545 to the measurement system 525 or the measurement library 530 (block 1153).

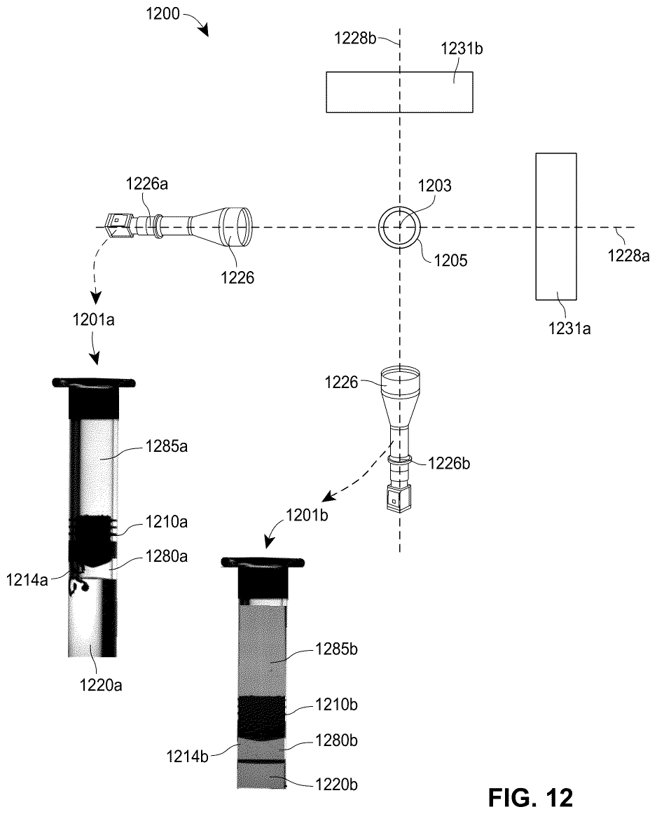

FIG. 12 is an AVI system 1200 including a syringe imaging device 1226 having a first camera 1226a and a second camera 1226b. The first camera 1226a includes a first central imaging axis 1228a aligned with a central syringe axis 1203 of a prefilled syringe 1205 and an illumination source 1231a. The first camera 1226a captures a first image 1201a of a portion of the prefilled syringe 1205 with the central imaging axis 1228a aligned with the central syringe axis 1203 from a fixed, predetermined rotational angle around the central syringe axis 1203. The first image 1201a may include liquid droplets and/or air bubbles at the junction of the plunger 1210a and syringe sidewall 1214a partially occlude the measurement point for air gap 1280a. Accordingly, the system 1200 may not measure a plunger depth 1285a and/or an air gap 1280a between the plunger 1210a and the fluid 1220a based on the first image 1201a.

The second camera 1226b includes a central imaging axis 1228b aligned with a central syringe axis 1203 of a prefilled syringe 1205 and an illumination source 1231b. The second camera 1226b captures a second image 1201b of a portion of the prefilled syringe 1205 with the central imaging axis 1228b aligned with the central syringe axis 1203 from a fixed, predetermined rotational angle around the central syringe axis 1203. The second image 1201b may not include liquid droplets and/or air bubbles at the junction of the plunger 1210b and syringe sidewall 1214b. The system 1200 may measure a plunger depth 1285b and/or an air gap 1280b between the plunger 1210b and the fluid 1220b based on the second image 1201b.

Although the systems, methods, devices, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

1. A computer-implemented method for measuring an air gap in a syringe having a plunger and containing a fluid, the method comprising:
capturing, using a syringe imaging device, a first image of at least a portion of the syringe from a first rotational angle around a central syringe axis of the syringe with respect to a first central imaging axis of the syringe imaging device;
capturing, using the syringe imaging device, a second image of at least a portion of the syringe from a second rotational angle around the central syringe axis with respect to a second central imaging axis of the syringe imaging device; and
determining, using one or more processors, an air gap measurement between the plunger and the fluid by analyzing at least the first image and the second image.

2. The method of claim 1, wherein the first and second images are captured by a first camera and a second camera, respectively, of the syringe imaging device, the first and second cameras have fixed orientations, the first camera has the first central imaging axis, and the second camera has the second central imaging axis.

3. The method of claim 1, wherein the first and second images are captured by moving a single camera of the syringe imaging device from the first rotational angle to the second rotational angle relative to the central syringe axis, the single camera has the first central imaging axis, and the second central imaging axis is the first central imaging axis.

4. The method of claim 1, wherein the first and second images are captured by a single camera of the syringe imaging device, and by rotating the syringe, wherein the single camera has the first central imaging axis, and wherein the second central imaging axis is the first central imaging axis.

5. The method of claim 1, wherein the air gap measurement includes a measurement associated with a bottom of a meniscus curve associated with a top surface of the fluid.

6. The method of claim 1, wherein the air gap measurement includes a measurement associated with an intersection of a perimeter of a bottom of the plunger and an inner surface of a syringe barrel.

7. The method of claim 1, wherein the air gap measurement includes a measurement of a difference between a bottom of a meniscus curve associated with a top surface of the fluid and an intersection of a perimeter of a bottom of the plunger and an inner surface of a syringe barrel.

8. The method of claim 1, further comprising:
determining, using the one or more processors, a plunger depth measurement by further analyzing at least the first image and the second image.

9. The method of claim 8, wherein the plunger depth measurement includes a measurement of a difference between a top of a syringe flange and a top of a plunger body.

10. The method of claim 1, further comprising:
providing a calibration standard having a predetermined air gap and a predetermined plunger depth;
inserting at least a portion of the calibration standard into a syringe;
capturing, using the syringe imaging device, a third image of at least a portion of the syringe and at least a portion of the calibration standard; and
calibrating, using the one or more processors, a measurement device based upon at least the third image.

11. A system for measuring an air gap in a syringe having a plunger and containing a fluid, the system comprising:
a syringe imaging device; and
one or more processors configured to:
control a syringe imaging device to capture a first image of at least a portion of the syringe from a first rotational angle around a central syringe axis of the syringe with respect to a first central imaging axis of the syringe imaging device;
control the syringe imaging device to capture a second image of at least a portion of the syringe from a second rotational angle around the central syringe axis with respect to a second central imaging axis of the syringe imaging device; and
determine an air gap measurement between the plunger and the fluid by analyzing at least the first image and the second image.

12. The system of claim 11, wherein the first and second images are captured by a first camera and a second camera, respectively, of the syringe imaging device, the first and second cameras have fixed orientations, the first camera has the first central imaging axis, and the second camera has the second central imaging axis.

13. The system of claim 11, wherein the first and second images are captured by moving a single camera of the syringe imaging device from the first rotational angle to the second rotational angle relative to the central syringe axis, the single camera has the first central imaging axis, and the second central imaging axis is the first central imaging axis.

14. The system of claim 11, wherein the first and second images are captured by moving a single camera of the syringe imaging device from the first rotational angle to the second rotational angle relative to the central syringe axis, the single camera has the first central imaging axis, and the second central imaging axis is the first central imaging axis.

15. The system of claim 11, further comprising a syringe rotation mechanism that includes a transparent tube syringe receptacle having an inner diameter larger than an outside diameter of the syringe.

16. The system of claim 11, wherein the syringe rotation mechanism is configured to hold the syringe with the central syringe axis in a vertical orientation.

17. The system of claim 11, further comprising:
a plurality of calibration standards with each calibration standard representing at least one of: a predetermined air gap or a predetermined plunger depth.

18. The system of claim 11, further comprising:
a data conversion device configured to convert the air gap measurement to an electronic batch record data format.

19. A non-transitory computer-readable medium storing computer-readable instructions that, when executed by one or more processors, causes the one or more processors to:
control a syringe imaging device to capture a first image of at least a portion of the syringe having a plunger and containing a fluid from a first rotational angle around a central syringe axis of the syringe with respect to a first central imaging axis of the syringe imaging device;
control the syringe imaging device to capture a second image of at least a portion of the syringe from a second rotational angle around the central syringe axis with respect to a second central imaging axis of the syringe imaging device; and
determine an air gap measurement between the plunger and the fluid by analyzing at least the first image and the second image.

20. The non-transitory computer-readable medium of claim 19, wherein the air gap measurement data is representative of at least one of: six different images, twelve different images, or twenty-four different images, wherein each different image includes a different portion of a perimeter surface of the syringe, and wherein the air gap measurement data includes different air gap measurement data based upon each different image.

21. The non-transitory computer-readable medium of claim 19, wherein further execution of the computer-readable instructions by the one or more processors causes the one or more processors to:
filter the air gap measurement data by deleting one or more highest air gap measurements and one or more lowest air gap measurements.

22. The non-transitory computer-readable medium of claim 20, wherein further execution of the computer-readable instructions by the one or more processors causes the one or more processors to:
generate an average air gap measurement that is based upon two or more air gap measurements.

23. The non-transitory computer-readable medium of claim 19, wherein further execution of the computer-readable instructions by the one or more processors causes the one or more processors to:
generate plunger depth measurement data based upon the syringe image data.

24. The non-transitory computer-readable medium of claim 19, wherein further execution of the computer-readable instructions by the one or more processors causes the one or more processors to:
receive calibration data that is representative of at least one of: a predetermined air gap measurement or a predetermined plunger depth measurement; and
calibrate a measurement device based on the calibration data.

25. The non-transitory computer-readable medium of claim 19, wherein the first and second images are captured by a first camera and a second camera, respectively, of the syringe imaging device, the first and second cameras have fixed orientations, the first camera has the first central imaging axis, and the second camera has the second central imaging axis.

26. The non-transitory computer-readable medium of claim 19, wherein the first and second images are captured by moving a single camera of the syringe imaging device from the first rotational angle to the second rotational angle relative to the central syringe axis, the single camera has the first central imaging axis, and the second central imaging axis is the first central imaging axis.

27. The non-transitory computer-readable medium of claim 19, wherein the first and second images are captured by moving a single camera of the syringe imaging device from the first rotational angle to the second rotational angle relative to the central syringe axis, the single camera has the first central imaging axis, and the second central imaging axis is the first central imaging axis.
